(19) United States
(12) Patent Application Publication
(10) Pub. No.: US 2013/0131520 A1
(43) Pub. Date: May 23, 2013

(54) BIOLOGICAL INFORMATION MEASURING APPARATUS AND BIOLOGICAL INFORMATION MEASURING METHOD

(71) Applicant: Seiko Instruments Inc., Chiba (JP)

(72) Inventor: Keisuke TSUBATA, Chiba (JP)

(73) Assignee: SEIKO INSTRUMENTS INC., Chiba (JP)

(21) Appl. No.: 13/676,398

(22) Filed: Nov. 14, 2012

(30) Foreign Application Priority Data
Nov. 21, 2011 (JP) 2011-254287

(51) Int. Cl. A61B 5/0205; A61B 5/0488; A61B 5/00

(52) U.S. Cl. 600/483; 600/300

(57) ABSTRACT

A biological information measuring apparatus includes: a measuring apparatus configured to measure biological information, and a display apparatus configured to switch a first frequency that receives first information indicating the biological information transmitted from the measuring apparatus and a second frequency which is a frequency that transmits second information generated on the basis of the received first information and is a frequency higher than the first frequency.

### BACKGROUND OF THE INVENTION

1. Technical Field

The present invention relates to a biological information measuring apparatus and a biological information measuring method.

2. Description of the Related Art

A biological information measuring apparatus includes, for example, a measuring unit configured to measure biological information, a display unit configured to display a measurement result measured by the measuring unit, and a data administration unit configured to add up and administrate the measurement result. In the biological information measuring apparatus configured in this manner, the information measured by the measuring unit is transmitted to the display unit, and the information received by the display unit is transmitted to the data administration unit. The transmission of information from the measuring unit to the display unit and the transmission of information from the display unit to the data administration unit is performed by using communication by means of radio of a band of 2.4 [GHz] (for example, see JP-A-2010-264247).

However, in the JP-A-2010-264247, since the display unit performs the transmission of information by using 2.4 [GHz], there is a problem that large power consumption is required for communication.

### SUMMARY OF THE INVENTION

It is an aspect of the present application to provide a biological information measuring apparatus and a biological information measuring method which can reduce power consumption required for transmission of information of the biological information measuring apparatus.

A biological information measuring apparatus according to an embodiment of the present application includes:

a measuring apparatus configured to measure biological information; and a display apparatus configured to switch a first frequency that receives first information indicating the biological information transmitted from the measuring apparatus and a second frequency which is a frequency that transmits second information generated on the basis of the received first information and is a frequency higher than the first frequency.

In the biological information measuring apparatus described above, preferably, the first frequency is a frequency lower than a legal radio communication frequency, and

the second frequency is a frequency used in a low electric power radio communication.

In the biological information measuring apparatus described above, preferably, the first information is information converted at the first frequency synchronously with the signal indicating the biological information.

In the biological information measuring apparatus described above, preferably, the first information is information including at least one of the heart rate and information indicating a muscle activity obtained by surface electromyography, and the second information includes information indicating heart rate generated on the basis of the first information or the muscle activity.

In the biological information measuring apparatus described above, the display device includes a measuring switch, and receipt of the first information is started when the measurement switch is pressed, and receipt of the first information is stopped when the measurement switch is pressed after the receipt of the first information has started.

According to an aspect of the present application, there is provided a biological information measuring method in a biological information measuring apparatus including: a time counting procedure to measure biological information by a measuring apparatus; and a display procedure to switch a first frequency that receives first information indicating the biological information transmitted from the measuring apparatus and a second frequency which is a frequency that transmits second information generated on the basis of the received first information and is a frequency higher than the first frequency.

In the present application, since the measuring apparatus uses the frequency which receives the first information indicating the measured biological information, which is lower than the frequency at which the second information on the basis of the received first information is transmitted, the biological information measuring apparatus and the biological information measuring method which can reduce power consumption required for transmission of information in the biological information measuring apparatus are provided.

### BRIEF DESCRIPTION OF THE DRAWINGS

FIG. 1 is a drawing for explaining a schematic configuration of a biological information measuring apparatus according to an embodiment;

FIG. 2 is a block diagram illustrating a measuring apparatus according to the embodiment;

FIG. 3 is a block diagram illustrating a display apparatus according to the embodiment;

FIG. 4 is a block diagram of a data administration apparatus according to the embodiment;

FIG. 5 is a sequence drawing of an example of a process of the biological information measuring apparatus according to the embodiment;

FIG. 6 is a drawing illustrating waveforms of respective portions of the measuring apparatus according to the embodiment;

FIG. 7 is an drawing illustrating a period from a time t1 to a time t30 in FIG. 6 in an enlarged scale; and

FIG. 8 is a drawing illustrating the waveforms of respective portions of a display apparatus according to the embodiment.

### DESCRIPTION OF THE PREFERRED EMBODIMENTS

Referring now to the drawings, the invention will be described in detail. The invention is not limited to the embodiment of the invention, and may be modified variously within a range of the technical ideas thereof.

### First Embodiment

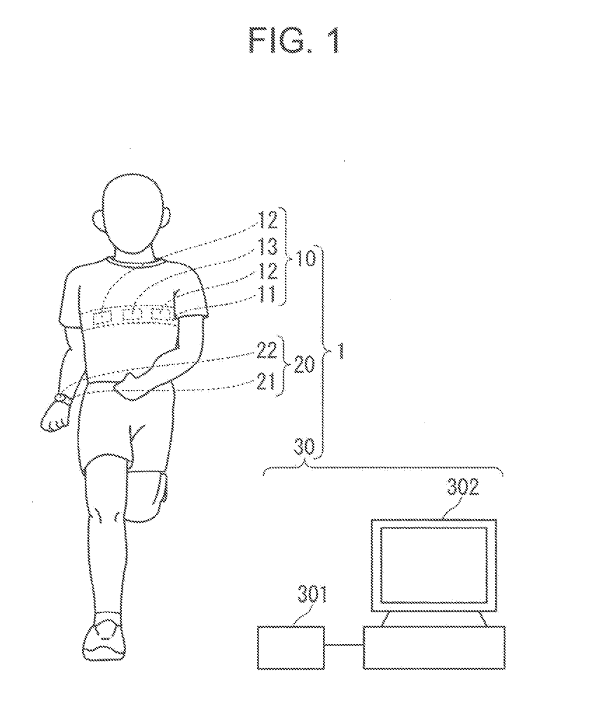

FIG. 1 is a drawing for explaining a schematic configuration of a biological information measuring apparatus 1 according to the invention.

As illustrated in FIG. 1, the biological information measuring apparatus 1 includes a measuring apparatus 10, a display apparatus 20, and a data administration apparatus 30. The measuring apparatus 10 includes a strap 11, an electrode 12, and a control unit 13. The display apparatus 20 is provided with a belt 21 and a display apparatus unit 22.

The strap 11 of the measuring apparatus 10 clips the measuring apparatus 10 on a chest portion of a test subject. The strap 11 includes two electrodes 12 with the control unit 13 interposed therebetween.

The measuring apparatus 10 measures biological information. Examples of the biological information include information indicating heart rates (hereinafter, referred to as heart rate information), information indicating cardiac actions. The biological information may be information obtained from muscle activities.

The belt 21 of the display apparatus 20 clips the display apparatus 20 to a wrist of the test subject. The display control unit 22 is attached to the belt 21.

The display apparatus 20 is configured to switch a first frequency that receives first information indicating biological information transmitted from the measuring apparatus 10 and a second frequency which is a frequency that transmits second information generated on the basis of the received first information and is a frequency lower than the first frequency.

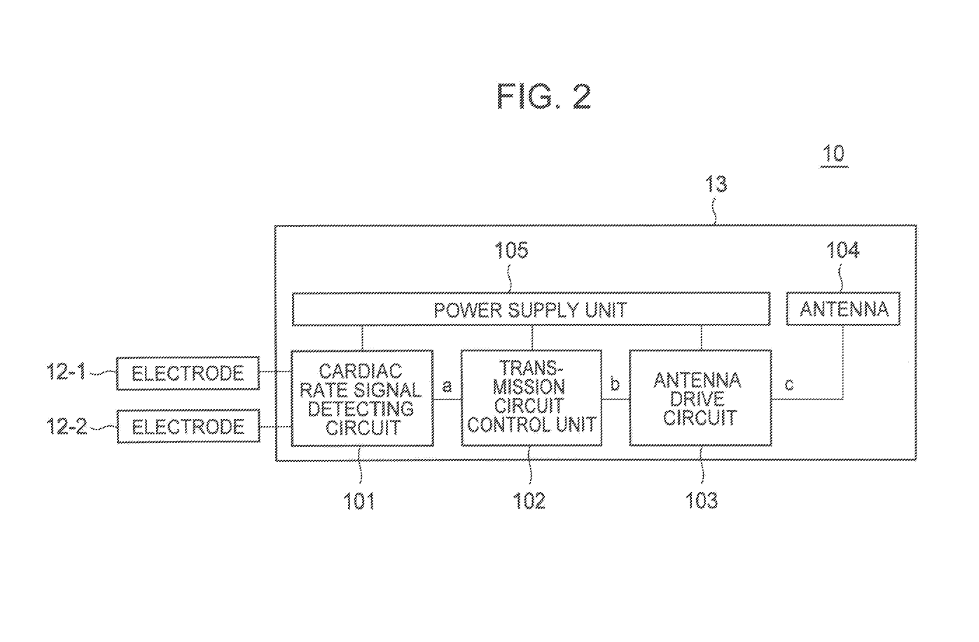

Subsequently, a configuration of the measuring apparatus 10 will be described with reference to FIG. 2.

FIG. 2 is a block diagram illustrating a measuring apparatus according to the embodiment. As illustrated in FIG. 2, the two electrodes 12 (12-1 and 12-2) are connected to the control unit 13 of the measuring apparatus 10. The control unit 13 is provided with a heart rate signal detecting circuit 101, a transmission circuit control unit 102, an antenna drive circuit 103, an antenna 104, and a power supply unit 105.

The electrodes 12 (12-1 and 12-2) of the measuring apparatus 10 detect a potential on the basis of the cardiac action, and output the detected potential to the heart rate signal detecting circuit 101 of the control unit 13. The potential detected by the too electrodes 12 is used for generating a signal indicating the muscle activity by the heart rate signal detecting circuit 101. Then, the signal indicating the cardiac action Is used by the display apparatus 20 for calculating the heart rate by calculating the frequency of the signal indicating the cardiac action.

The heart rate signal detecting circuit 101 generates a signal a indicting a timing of the heart rate on the basis of the potential difference output from the electrode 12-1 and the electrode 12-2, and the generated signal a is output to the transmission circuit control unit 102.

The transmission circuit control unit 102 generates a signal b on the basis of the signal a output from the heart rate signal detecting circuit 101, and outputs the generated signal b to the antenna drive circuit 103.

The antenna drive circuit 103 converts the signal b output from the transmission, circuit control unit 102 into a burst signal and transmits a converted signal c via the antenna 104. Specifically, the antenna drive circuit 103 transmits the signal c via the antenna 104 at, for example, 5 [KHz] (first frequency).

The power supply unit 105 supplies power to the heart rate signal detecting circuit 101, the transmission circuit control unit 102, and the antenna drive circuit 103.

In other words, the measuring apparatus 10 transmits the biological information (first information) at the first frequency.

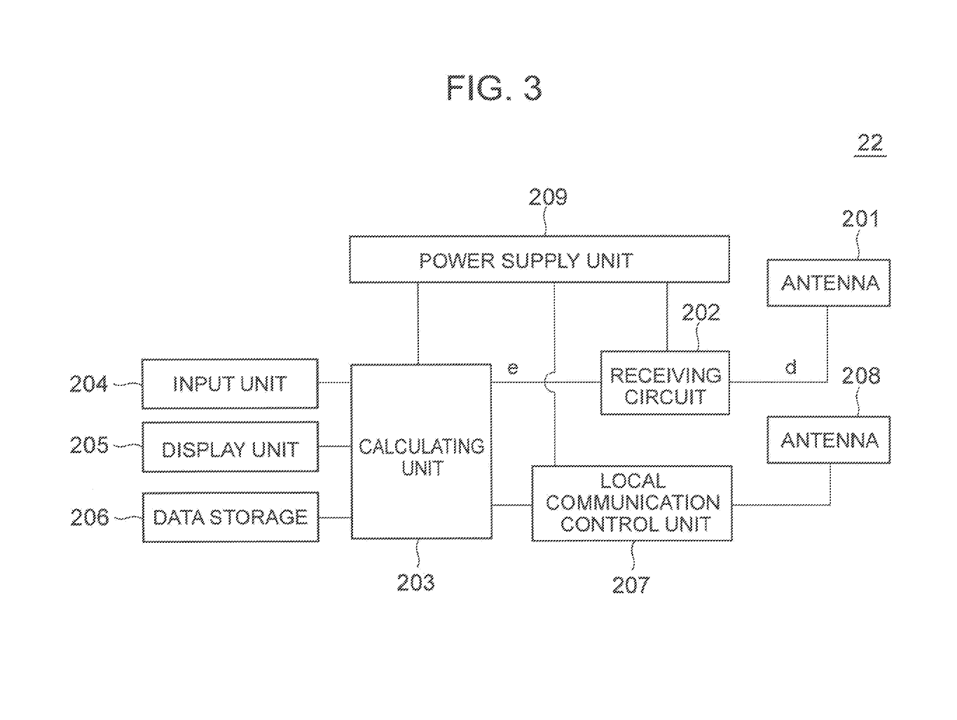

Subsequently, a configuration of the display apparatus 20 will be described with reference to FIG. 3.

FIG. 3 is a block diagram of the display apparatus 20 according to the embodiment. As illustrated in FIG. 3, the display control unit 22 of the display apparatus 20 includes an antenna 201, a receiving circuit 202, a calculating unit 203, an input unit 204, a display unit 205, a data storage 206, a local communication control unit 207, an antenna 208, and a power source unit 209.

The receiving circuit 202 receives a signal d transmitted by the measuring apparatus 10 via the antenna 201. The receiving circuit 202 performs wave shaping on the received signal d, and outputs a wave-shaped signal as a signal e to the calculating unit 203.

The calculating unit 203 calculates the heart rate per minute on the basis or the signal e output from the receiving circuit 202, and displays the calculated heart rate information on the display unit 205. The calculating unit 203 correlates the calculated heart rate information to the information indicating the date and time when the heart rate is calculated and stores the same in the data storage 206. The calculating unit 203 detects the fact that the communication of the local communication control unit 207 with respect to the data administration apparatus 30 is established, and then reads out the second information stored in the data storage 206, and outputs the read second information to the local communication control unit 207.

The input unit 204 is provided with a measuring switch, not shown. When the display unit 205 is a touch panel, the display unit 205 may be configured to function as the input unit 204.

The display unit 205 is, for example, a liquid crystal panel and an organic EL (Electro Luminescence) panel.

The heart rate information and the information indicating the data and time when the heart rate is calculated are correlated and stored in the data storage 206.

The local communication control unit 207 detects the fact that the display apparatus 20 is placed on a data transmitting unit 301 of the data administration apparatus 30 via the antenna. When the fact that the display apparatus 20 is placed on the data transmitting unit 301 is detected, the local communication control unit 207 outputs the result of detection to the calculating unit 203.

The local communication control unit 207 converts the second information that the calculating unit 203 outputs into a format of a prescribed communication standard and transmits the converted information via the antenna 208. The transmitted information is text data indicating, for example, the heart rate and the time and date when the heart rate is calculated.

The local communication control unit 207 performs communication with the data administration apparatus 30 using Low Electric Power Ratio Communication Standard of NFC (Near Field Communication). The local communication control unit 207 uses a carrier frequency (second frequency) of, for example, 13.56 [MHz].

The power source unit 201 supplies power to the receiving circuit 202, the calculating unit 203, the input unit 204, the display unit 205, the data storage 206, and the local communication control unit 207.

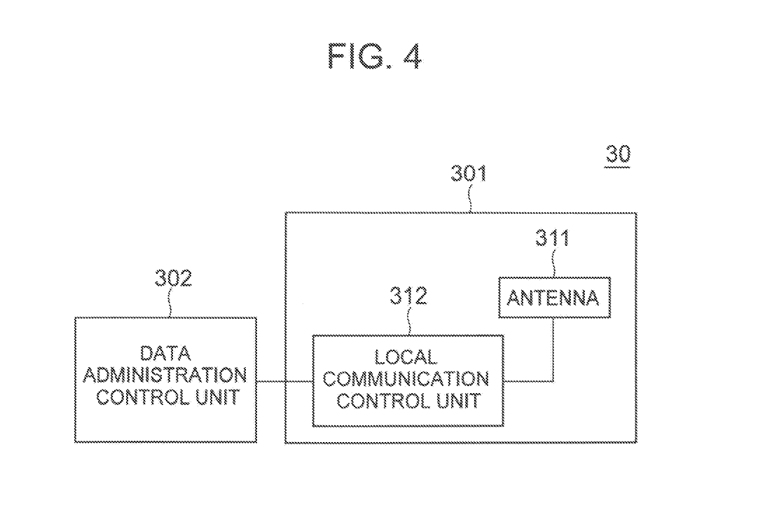

Subsequently, the configuration of the data administration apparatus 30 will be described with reference to FIG. 4.

FIG. 4 is a block diagram of a data administration apparatus according to the embodiment.

As illustrated in FIG. 4, the data administration apparatus 30 is provided with the data transmitting unit 301 and a data administration control unit 302. The data transmitting unit 301 is provided with an antenna 311 and a local communication control unit 312.

The data administration apparatus 30 performs information transmission with respect to the display apparatus 20, and administrates the transmitted information.

The local communication control unit 312 of the data transmitting unit 301 performs the information transmission with the display apparatus 20 via the antenna 311 by communication under the Low Electric Power Radio Communication Standard of NFC. The local communication control unit 312 outputs the transmitted information to the data administration control unit 302.

The data administration control unit 302 receives the information output, from the local communication control unit 312 and stores the received information in a storage. The data administration control unit 302 displays the heart rate information every week, or every month depending on an instruction from a user on a display on the basis of the information stored in the storage. The data administration control unit 302 may foe composed of a personal computer, a display, a printer, and the like.

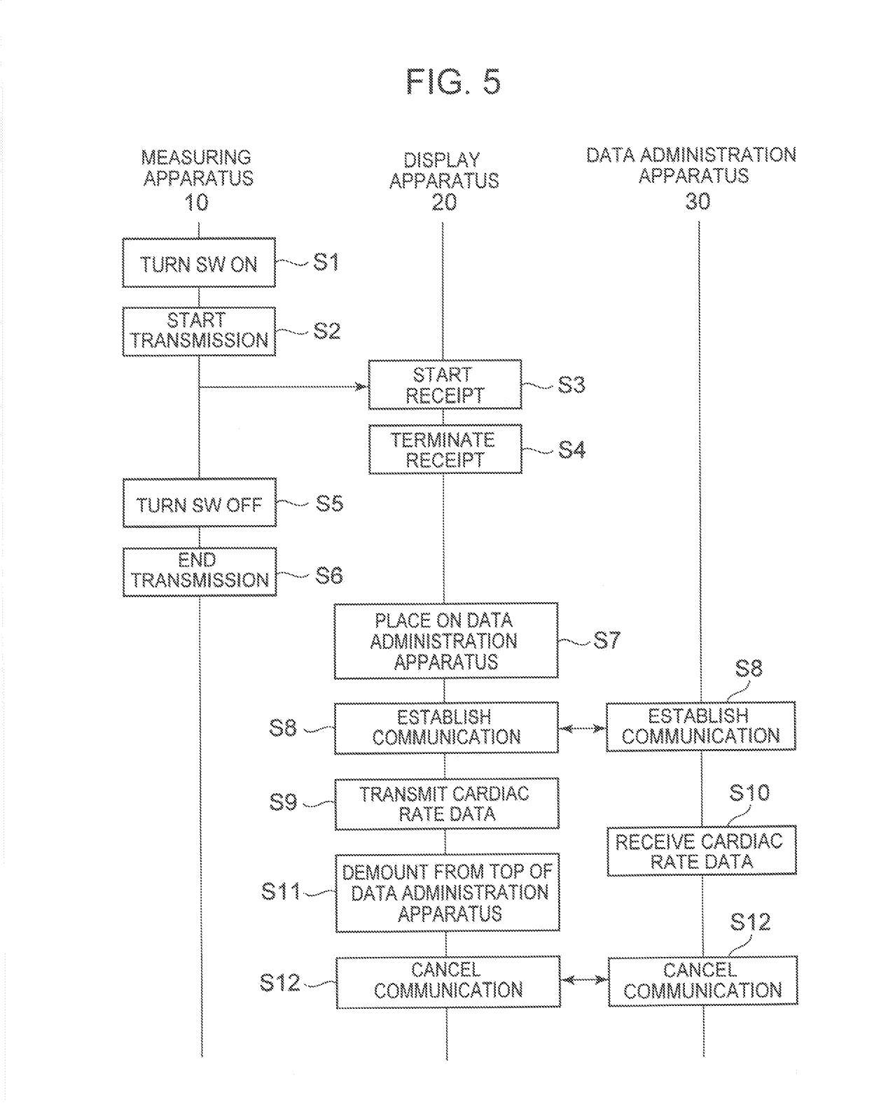

Subsequently, an example of a procedure of process performed by the biological information measuring apparatus will be described with reference to FIG. 5.

FIG. 5 is a sequence drawing of an example of a process of the biological information measuring apparatus according to the embodiment.

(Step S1) The user wears the measuring apparatus 10 on his or her body, turns ON a power supply switch (not shown) provided on the control unit 13 of the measuring apparatus 10, and starts measurement. For example, before the user performs an exercise, the user wears the measuring apparatus 10, turns ON a power supply and starts measurement.

(Step S2) The measuring apparatus 10 starts

transmission of the signal c on the basis of the measured heartbeat information. Subsequently, the measuring apparatus 10 performs transmission of the signal c at timings when the potential difference between the electrodes 12 is detected. For reference, the transmission, is performed until the power supply switch of the control unit 13 is pressed by the user and the measurement is terminated.

(Step S3) When the measurement switch of the input unit 204 is pressed, the receiving circuit 202 of the display apparatus 20 starts receiving of the signal c transmitted by the measuring apparatus 10 via the antenna 201.

Subsequently, the display apparatus 20 performs receiving of the signal transmitted by the measuring apparatus 10. The receipt of the signal continues until the measurement switch of the input unit 204 is pressed. While the user performs the exercise, for example, the display apparatus 20 continues the receipt of the signal measured by the measuring apparatus 10. The calculating unit 203 of the display apparatus 20 calculates the heart rate per minute every minute, for example, on the basis of the signal e output from the receiving circuit 202. The calculating unit 203 determines the time when the calculation is performed as the measured time, correlates the time of measurement with the calculated heart rate information, and stores the same in the data storage 206.

(Step S4) When the measurement switch of the input unit 204 is pressed, the display apparatus 20 terminates receiving of the signal transmitted by the measuring apparatus 10.

(Step S5) The user turns the power supply switch provided on the control unit 13 of the measuring apparatus 10 into an OFF state.

(Step S6) The measuring apparatus 10 detects the fact that the power supply switch is pressed, and terminates the measurement according to the result of detection. For example, after having terminated the exercise, the user brings the power supply switch provided on the control unit 13 to the OFF state to terminate the measurement.

(Step S7) The user places the display apparatus 20 on the data transmitting unit 301 of the data administration apparatus 30 for transmitting the measured information to the data administration apparatus 30.

(Step S8) The data administration apparatus 30 detects the fact that the display apparatus 20 is placed on the data transmitting unit 301. On the basis of the result of detection, the data administration apparatus 30 establishes the communication with the display apparatus 20.

(Step S9) The local communication control unit 207 of the display apparatus 20 starts transmission of the heart rate information (second information) at every time read out by the calculating unit 203 from the data storage 206 to the data administration apparatus 30 via the antenna 208.

(Step S10) The local communication control unit 312 of the data administration apparatus 30 receives the second information transmitted by the display apparatus 20 via the antenna 311.

(Step S11) After having completed the transmission of the second information, the user demounts the display apparatus 20 from the top of the data transmitting unit 301 of the data administration apparatus 30. Completion of the transmission of the second information from the display apparatus 20 is transmitted, for example, by transmitting the fact that the receiving of the second information is completed from the data administration apparatus 30 to the display apparatus 20. The display apparatus 20 displays the fact that the transmission of the second information is completed on the display unit of the display apparatus 20 on the basis of information indicating the fact that the receipt of the second information received from the data administration apparatus 30 has completed. The user may confirm the fact chat the transmission, of the second information is completed by confirming this display.

(Step S12) The data administration apparatus 30 detects the fact that the display apparatus 20 is demounted from the top of the data transmitting unit 301, and cancels the communication with respect to the display apparatus on the basis of the result of detection.

The process of the biological information measuring apparatus will be terminated in the procedure as described thus far.

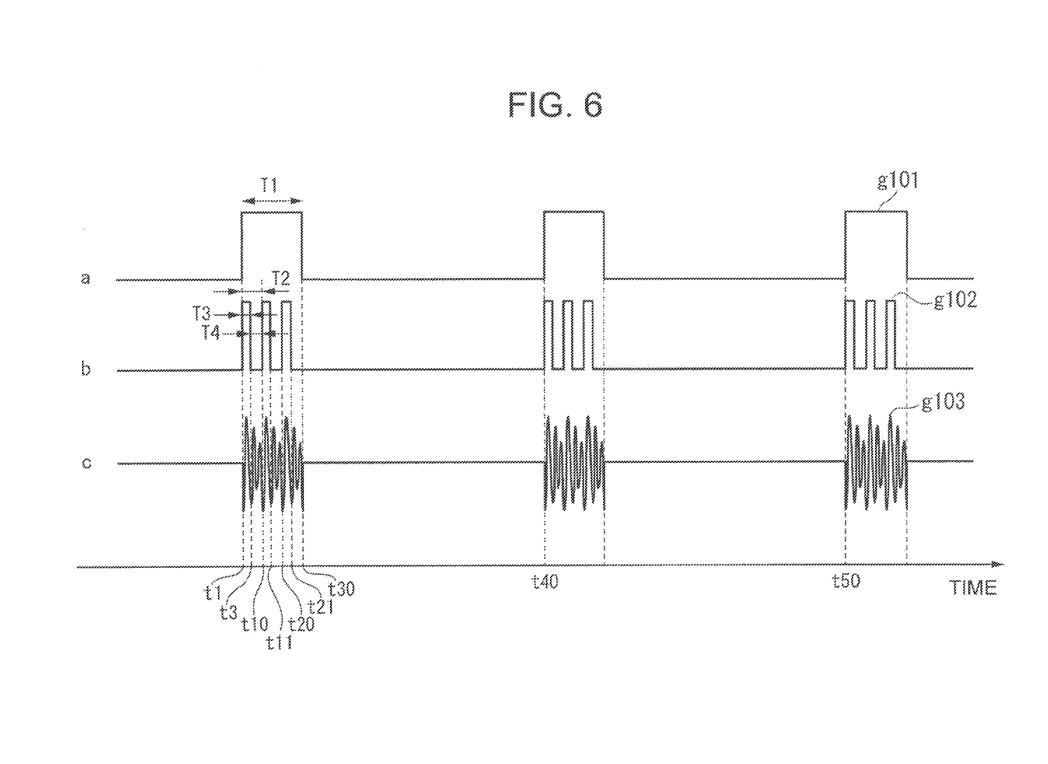

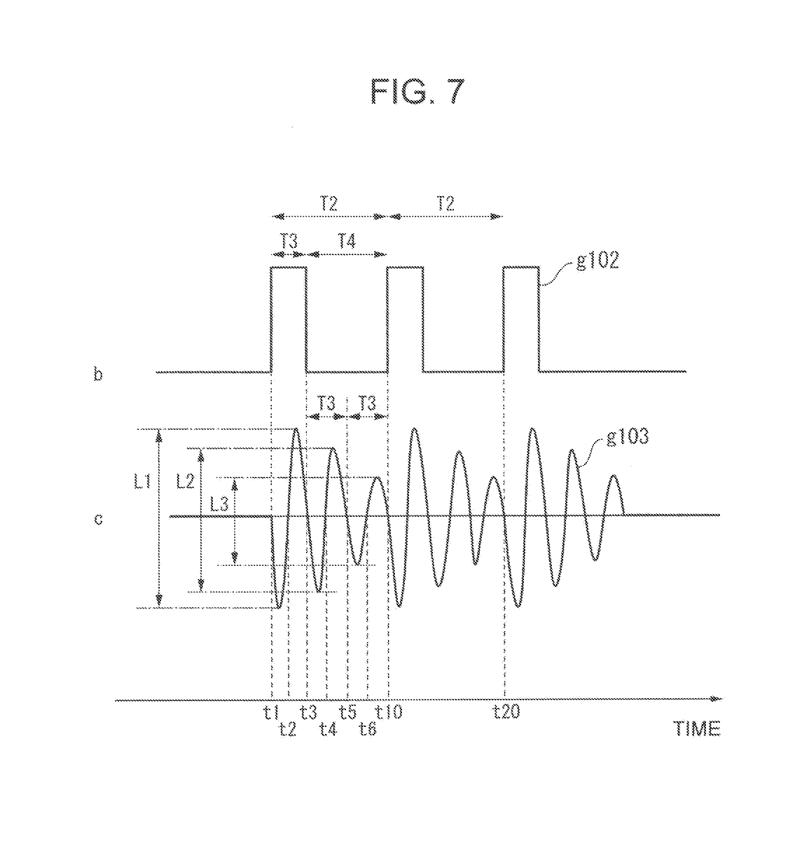

Subsequently, the transmission of the signal from the measuring apparatus 10 to the display apparatus 20 will be described with reference to FIG. 2, FIG. 6, and FIG. 7.

FIG. 6 is a drawing illustrating waveforms of the respective portions of the measuring apparatus 10 according to the embodiment. FIG. 7 is a drawing illustrating a period from a time t1 to a time t30 in FIG. 6 illustrating in an enlarged scale. FIG. 7 is a drawing illustrating waveforms of the respective portions of the display apparatus 20 according to the embodiment.

In FIG. 6 and FIG. 7, the lateral axis indicates the time and the vertical axis indicates levels of the respective waveforms. In FIG. 5, a waveform g101 is a waveform of the signal a output from the heart rate signal detecting circuit 101. A waveform g102 is a waveform of the signal b output from the transmission circuit control unit 102, and a waveform g103 is a waveform of the signal c output from the antenna drive circuit 103.

In FIG. 6, the lateral axis indicates the time and the vertical axis indicates levels of the respective waveforms. Also, the waveform g102 is the waveform of the signal b output from the transmission circuit control unit 102, and the waveform g103 is the waveform of the signal c output from the antenna drive circuit 103.

As illustrated in FIG. 6, the heart rate signal detecting circuit 101 generates the high-level signal a for a period from the time t1 to t30 (T1) when the potential difference between two electrodes 12-1 and 12-2 is larger than a predetermined threshold value is detected. Also, the heart rate signal detecting circuit 101 generates the low level signal a for a period (from the time t30 to t40) when the fact that the potential difference between the two electrodes 12-2 and 12-2 is smaller than the predetermined threshold value is detected. The heart rate signal detecting circuit 101 outputs the generated signal a (the waveform g101) to the transmission circuit control unit 102.

Subsequently, the transmission circuit control unit 102 generates the signal b of a cycle T2 (the time t10 to t1) during the period T1 at a timing of rising of the signal a (waveform g101) output from the heart rate signal detecting circuit 101. The transmission circuit control unit 102 outputs the generated signal b to the antenna drive circuit 103. As illustrated in FIG. 5, the waveform g102 is in a high level during a period (T3) from the time t1 to the time t3, and in a low level during a period (T4) from the t3 to t10.

Subsequently, as illustrated in FIG. 6 and FIG. 7, the antenna drive circuit 103 generates the waveform g103 having a signal amplitude L3 for a cycle T3 of the waveform g103 at a rising timing (time t1) of the signal b (waveform g102) output from the transmission circuit control unit 102.

Subsequently, the antenna drive circuit 103 stops the generation of the waveform at a dropping timing (time t3) of the signal b (waveform g102) output from the transmission circuit control unit 102. Therefore, in the waveform g103, the waveform during a period from the time t3 to the time t5 and the waveform during a period from t5 to t10 are kept while the waveform during the period from the time t1 to t3 is attenuated in the transmission path. In this manner, since the antenna drive circuit 103 is configured to generate the waveform only during the period from the time t1 to t3, power consumption of the display apparatus 20 can further be alleviated.

The antenna drive circuit 103 transmits the signal c (waveform g103) generated in this manner via the antenna 104.

As illustrated in FIG. 7, the waveform g103 includes three signals from a first to a third signals at the cycle T3 during the period of the cycle T1. The first signal is a signal having the signal amplitude L1 during a period from the time t1 to the time t3. The second signal is a signal having a signal amplitude L2 during a period from the time t3 to the time t5. The third signal is a signal having a signal amplitude L3 during a period from the time t5 to the time t10.

The signal amplitude L1 is larger than the signal amplitudes L2 and L3. The signal amplitude L2 is larger than the signal amplitude L3 and smaller than the signal amplitude L1. The signal amplitude L3 is smaller than the signal amplitudes L1 and L2. The cycle T3 is a carrier wave frequency from the measuring apparatus 10 to the display apparatus 20, and is, for example, 5 [kHz].

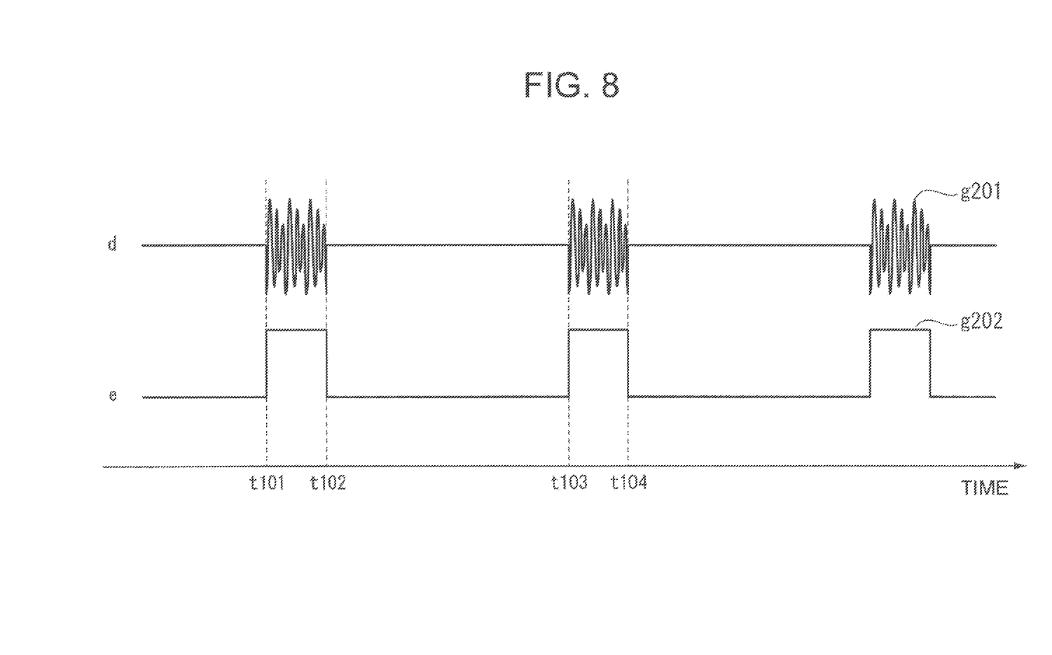

Subsequently, an example of a transmission process of the display apparatus 20 will be described with reference to FIG. 8.

FIG. 8 is a drawing illustrating waveforms of the respective portions of the display apparatus 20 according to the embodiment. In FIG. 8, the lateral axis indicates the time and the vertical axis indicates levels of the respective waveforms. Also, a waveform g201 is a waveform of the signal d input to the receiving circuit 202, and a waveform g202 is a waveform of the signal e output from the receiving circuit 202.

The receiving circuit 202 starts receiving of the signal transmitted by the measuring apparatus 10 via the antenna 201 when the measurement switch of the input unit 204 is pressed. The receiving circuit 202 generates the high-level signal e during a period (time t101 to t102) when an absolute value of the received signal d (waveform g201) is larger than the prescribed threshold value. The receiving circuit 202 generates the low-level signal e during a period (time t102 to t103) when the absolute value of the received signal d (waveform g201) is smaller than the prescribed threshold value. The receiving circuit 202 outputs the generated signal e to the calculating unit 203.

As illustrated in FIG. 8, the signal d generated by the receiving circuit 202 (waveform, g202) is equivalent to the signal a (waveform g101) illustrated in FIG. 6.

As illustrated in FIG. 8, the waveform g202 is in a high level during the period from the time t101 to t102, and is in the low level from the time t102 to the time t103.

The calculating unit 203 counts a period during the rising (time t101) of the signal e and a rising (time t103) of the same output from the receiving circuit 202 using a counter in the interior thereof. The calculating unit 203 calculates, for example, the heart rate for one minute on the basis of the counted value. The calculating unit 203 displays the calculated heart rate information on the display unit 205, and further stores the same in the data storage 206.

The user presses the measuring switch of the input unit 204 after the heart rate is displayed on the display unit 205. The calculating unit 203 detects the fact that the measuring switch is pressed, and terminates receipt of the signal transmitted by the measuring apparatus 10 according to the result of detection.

As described above, since the measuring apparatus 10 uses the frequency which receives the first information indicating the measured biological information, which is lower than the frequency at which the second information on the basis of the received first information is transmitted, power consumption required for transmitting the information may be reduced. Also, the display apparatus 20 is configured to receive the first information from the measuring apparatus 10 at the first frequency, and switch the second information to the second frequency and transmits the same to the data administration apparatus 30, power consumption required for transmitting the information may be reduced.

Since the frequency of 5 [KHz] is used for communication between the measuring apparatus 10 and the display apparatus 20, approbation of this frequency is not needed.

Furthermore, the communication between the display apparatus 20 and the data administration apparatus 30 is performed on the basis of the Low Electric Power Radio Communication Standard, the display apparatus 20 and the data administration apparatus 30 can get closer at the time of transmission of information, the influence of exogenous noise is advantageously reduced. As in the related art, pairing or encryption or the like required for performing the communication at a wireless standard of 2.4 [GHz] band is no longer necessary, the configuration of the display apparatus 20 can be simplified. Since the communication between the display apparatus 20 and the data administration apparatus 30 is performed on the basis of Low Electric Power Radio Communication Standard, the display apparatus 20 does not perform the transmission of information with respect to the data administration apparatus 30 until being placed on the data transmitting unit 301 of the data administration apparatus 30. Consequently, power consumption at the time when the information transmission is not performed may be reduced.

In this embodiment, although an example of 5 [KHz] as an example of the frequency that the measuring apparatus 10 transmits to the display apparatus 20, a frequency lower than the 10 [KHz], which is Low Electric Power Radio Communication Standard, may be used. The Low Electric Power Radio Communication Standard is a frequency band which requires approbation.

Although an example in which the calculating unit 203 correlates the heart rate information with the date and time calculated by the calculating unit 203 and stores the same in the data storage 206 has been described in this embodiment, the data and time when the heart rate information is detected may be correlated before storage.

In this embodiment, as illustrated in FIG. 7, an example in which the signal c transmitted from the measuring apparatus 10 to the display apparatus 20 (waveform g103) generates only the waveform of the signal amplitude L1 from the time t1 to t3, and signals which are not attenuated within the transmission channel are used for other amplitudes L2 and L3 has been described. However, the invention is not limited thereto. In FIG. 7, the waveform during a period (T2) from the time t1 to the t10 many be of a period corresponding to two cycles or three cycles or more according to the frequency or the signal amplitude L1 generated by the antenna drive circuit 103. According to the frequency and the signal amplitude L1 generated by the antenna drive circuit 103, the period T2 of the signal b generated by the transmission circuit control unit 102 may be adjusted. For example, when the display apparatus 20 is designed so that the attenuation rate of the signal amplitude L1 of the signal c (waveform, g103) is low, the transmission circuit control unit 102 may be configured to generate a high-level signal only during the period from the time t1 to t3. Then, in this manner, the antenna drive circuit 103 may be configured to generate only the signal having the amplitude L1 at the timing of the high-level signal generated by the transmission circuit control unit 102 and use only the attenuation signal having the amplitude L1 during the period from the time t3 to the time t30.

The function of the display apparatus 20 in the embodiment may be provided in a wristwatch.

What is claimed is:

1. A biological information measuring apparatus comprising:
a measuring apparatus configured to measure the biological information; and
a display apparatus configured to switch a first frequency that receives first information indicating the biological information transmitted from the measuring apparatus and a second frequency which is a frequency that transmits second information generated on the basis of the received first information and is a frequency higher than the first frequency.

2. The biological information measuring apparatus according to claim 1, wherein
the first frequency is a frequency lower than a legal radio communication frequency, and
the second frequency is a frequency used in a low electric power radio communication.

3. The biological information measuring apparatus according to claim 1, wherein the first information is information converted at the first frequency synchronously with the signal indicating the biological information.

4. The biological information measuring apparatus according to claim 2, wherein the first information is information converted at the first frequency synchronously with the signal indicating the biological information.

5. The biological information measuring apparatus according to claim 1, wherein the first information is information including at least one of the heart rate and information indicating a muscle activity obtained by surface electromyography, and
the second information includes information indicating heart rate generated on the basis of the first information or the muscle activity.

6. The biological information measuring apparatus according to claim 2, wherein the first information is information including at least one of the heart rate and information indicating a muscle activity obtained by surface electromyography, and
the second information includes information indicating heart rate generated on the basis of the first information or the muscle activity.

7. The biological information measuring apparatus according to claim 3, wherein the first information is information including at least one of the heart rate and information indicating a muscle activity obtained by a surface electromyography, and
the second information includes information indicating heart rate generated on the basis of the first information or the muscle activity.

8. The biological information measuring apparatus according to claim 4, wherein the first information is information including at least one of the heart rate and information indicating a muscle activity obtained by surface electromyography, and
the second information includes information indicating heart rate generated on the basis of the first information or the muscle activity.

9. The biological information measuring apparatus according to claim 1, wherein the display device includes a measuring switch, and
receipt of the first information is started when the measurement switch is pressed, and receipt of the first information is stopped when the measurement switch is pressed after the receipt of the first information has started.

10. The biological information measuring apparatus according to claim 2, wherein the display device includes a measuring switch, and
receipt of the first information is started when the measurement switch is pressed, and receipt of the first information is stopped when the measurement switch is pressed after the receipt of the first information has started.

11. The biological information measuring apparatus according to claim 3, wherein the display device includes a measuring switch, and
receipt of the first information is started when the measurement switch is pressed, and receipt of the first information is stopped when the measurement switch is pressed after the receipt of the first information has started.

12. The biological information measuring apparatus according to claim 4, wherein the display device includes a measuring switch, and
receipt of the first information is started when the measurement switch is pressed, and receipt of the first information is stopped when the measurement switch is pressed after the receipt of the first information has started.

13. The biological information measuring apparatus according to claim 5, wherein the display device includes a measuring switch, and
receipt of the first information is started when the measurement switch is pressed, and receipt of the first information is stopped when the measurement switch is pressed after the receipt of the first information has started.

14. The biological information measuring apparatus according to claim 6, wherein the display device includes a measuring switch, and
receipt of the first information is started when the measurement switch is pressed, and receipt of the first information is stopped when the measurement switch is pressed after the receipt of the first information has started.

15. The biological information measuring apparatus according to claim 7, wherein the display device includes a measuring switch, and
receipt of the first information is started when the measurement switch is pressed, and receipt of the first information is stopped when the measurement switch is pressed after the receipt of the first information has started.

16. The biological information measuring apparatus according to claim 8, wherein the display device includes a measuring switch, and
receipt of the first information is started when the measurement switch is pressed, and receipt of the first information is stopped when the measurement switch is pressed after the receipt of the first information has started.

17. A biological information measuring method in a biological information measuring apparatus comprising:
a time counting procedure to measure biological information by a measuring apparatus; and
a display procedure to switch a first frequency that receives first information indicating the biological information transmitter from the measuring apparatus and a second frequency which is a frequency that transmits second information generated on the basis of the received first information and is a frequency higher than the first frequency.
